(19) United States
(12) Patent Application Publication
(10) Pub. No.: US 2005/0192349 A1
(43) Pub. Date: Sep. 1, 2005

(54) Isolation of the peroxisome proliferation-activated receptor-gamma (PPARgamma) ligand and methods of use thereof

(75) Inventors: Jeffrey S. Flier, West Newton, MA (US); Hui Fang, Tokyo (JP); Mario Ollero, Paris (FR); Anthony N. Hollenberg, Newton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Beth Israel Deaconess Medical Center, Inc., Boston, MA (US)

(21) Appl. No.: 11/017,417

(22) Filed: Dec. 20, 2004

Related U.S. Application Data
(63) Continuation of application No. PCT/US02/19738, filed on Jun. 20, 2002.

(51) Int. Cl. A61K 31/225; C12P 7/64

(52) U.S. Cl. 514/547; 435/134

(57) ABSTRACT

The present invention is drawn toward novel endogenous PPARγ ligand, the isolation of the ligand and the use of the isolated ligand to stimulate PPARγ activity in cells of interest. The invention is also drawn to diagnostic methods to detect the level of the ligand in a sample of interest.

### RELATED APPLICATION

This application is a continuation of International Application No. PCT/US2002/019738, which designated the United States and was filed 20 Jun. 2002, published in English. The entire teachings of the above application is incorporated herein by reference.

### GOVERNMENT SUPPORT

The invention was supported, in whole or in part, by a grant NIH NIDDK R01 DK46930 from the National Institutes of Health. The Government has certain rights in the invention.

### BACKGROUND OF THE INVENTION

The prevalence of obesity and diseases associated with obesity have increased rapidly in western society and to a lesser extent in the rest of the world. Obesity has been linked to many serious health problems, including insulin resistance, type 2 diabetes, cardiovascular disease, and hypertension. According to recent statistics, over half of all adults are considered overweight, and 7% have type 2 diabetes (Wilson et al., Annu. Rev. Biochem., 70:341-367 (2001)). As a consequence, the development and homeostasis of adipose tissue in mammals has become the subject of intense investigation.

The nuclear hormone receptor, peroxisome proliferator-activated receptor γ (PPARγ), is a transcription factor that is necessary for adipogenesis (Rosen et al., Mol. Cell 4:611-617 (1999)). PPARγ heterodimerizes with the retinoid X receptor (RXR) and regulates a number of genes by binding to specific response elements. PPARγ activation results in adipocyte differentiation from pre-adipocytes in vitro and in vivo. Transcription of adipocyte specific genes is increased upon PPARγ activation, including aP2, phosphoenol pyruvate carboxykinase, acyl CoA synthase, fatty acid translocase/CD36, and fatty acid transport protein-1 (see Rocchi and Auwerx, British Journal of Nutrition, 84(2):S223-S227 (2000)).

In humans, PPARγ has three isoforms, PPARγ1, PPARγ2, and PPARγ3 (see Fajas et al., Nutr. Metab. Cardiovasc. Dis., 11:64-69 (2001)). The PPARγ isoforms are splice variants, where the γ1 and γ3 variants are the same protein and the γ2 variant has 28 additional amino acids at the amino terminus (Id.). PPARγ is expressed in adipose tissue, large intestine and hematopoietic cells and to a lesser extent in kidney, liver and small intestine. PPARγ is also expressed in muscle and PPARγ3 is expressed in macrophages (Id.).

It is thought that the natural ligand for PPARγ is a Prostaglandin J2 derivative (15-deoxyΔ^{12,14}PG J2), (see Spiegelman, Diabetes, 47:507-514). In addition, certain polyunsaturated fatty acids, such as linoleic acid, have been shown to bind directly to PPARγ (Id.). However, unlike other known nuclear receptors whose dissociation constant for endogenous ligand is in the low nmol/l range, PPARγ has a very low affinity for 15-deoxyΔ^{12,14}PG J2 and the polyunsaturated fatty acids (2-50 μmol/l range). Therefore, it is unclear if 15-deoxyΔ^{12,14}PG J2 and the polyunsaturated fatty acids are present in sufficient quantities to activate PPARγ in vivo, due to their low affinity.

While PPARγ is known to be required for adipogensis, it affects the other cell types in which it is expressed. PPARγ is expressed in human peripheral blood monocytes and is induced by agents that induce macrophage differentiation. Therefore, PPARγ is thought to affect macrophage differentiation (see Fajas et al.). PPARγ is highly expressed in macrophage foam cells and atherosclerotic lesions, indicating that PPARγ plays a role in inflammatory diseases such as cardiovascular disease. Furthermore, PPARγ is highly expressed in colon epithelial cells and treatment of mice with synthetic PPARγ agonist reduces colon inflammation (see Fajas et al.). In addition, in rodents troglitazone, a synthetic PPARγ agonist, has been shown to inhibit smooth muscle cell proliferation and decrease the intima and media thickness of carotid arteries (see Barbier et al. Arterioscler. Thromb. Vasc. Biol., 22:717-726 (2002)).

PPARγ has also been shown to induce apoptosis in certain cancer cells or to induce certain cells to stop dividing. Modulation of PPARγ activity may be particularly useful in treating colon cancer. For example, synthetic PPARγ agonist has been shown to induce apoptosis in HT-29 colon cancer cells (Shimada et al. Gut, 50:658-664 (2002)). In addition, loss-of-function mutations in PPARγ have been found to be associated with primary sporadic colorectal cancer in humans, indicating that PPARγ activity may protect against development of colon cancer (Sarraf et al., Molecular Cell, 3:799-804 (1999)).

PPARγ activation has been studied in other cell types and cancers known to express PPARγ. PPARγ has been shown to be highly expressed in human primary and metastatic breast cancers and synthetic PPARγ agonist has been shown to induce terminal differentiation in cultured human breast cancer cells (Mueller et al., Molecular Cell, 1:465-470 (1998)). PPARγ has been shown to be expressed in liposarcoma at levels found in normal adipose tissue. Human liposarcoma cells have been shown to be induced to undergo terminal differentiation into adipocytes in vitro, upon treatment with micromolar amounts of synthetic PPARγ agonist (Tontonoz et al., Proc. Natl. Acad. Sci. USA, 94:237-241 (1997)). Furthermore, treatment of liposarcoma in vivo with synthetic PPARγ agonist has been shown to induce terminal differentiation and a reduction in cell proliferation in the tumors (Demitri et al., Proc. Natl. Acad. Sci. USA, 96:3951-3956 (1999)).

Synthetic anti-diabetic PPARγ agonists, such as thiazolidinediones (TZD) reduce insulin resistance in mice and humans (Rosen et al. and Rocchi and Auwerx). The synthetic PPARγ agonists also increase lipolysis of triglycerides in very low density lipoproteins (VLDL), (Rocchi and Auwerx). While TZDs are used as anti-diabetic drugs in humans, resulting in reduced insulin resistance, TZDs have some potential side effects that require monitoring. Certain TZDs at high does cause an increase in adipose cell formation in the bone marrow of rodents by causing adipogensis in the bone marrow stromal cells (Gimble et al., Mol. Pharmacol., 50:1087-1094 (1996). In addition, treatment with troglitazone resulted in liver toxicity in some patients (Spiegelman, Diabetes, 47:507-514). Furthermore, because thiazolidinediones increase lipolysis of triglycerides in VLDL, VLDL can be converted into LDL, and in fact, troglitazone and rosiglitazone are associated with a rise in LDL levels (see Fajas et al., Nutr. Metab. Cardiovasc. Dis., 11:64-69 (2001)).

### SUMMARY OF THE INVENTION

Despite all that is known about PPARγ, its pivotal role in adipogensis and as a target for reducing insulin resistance, the high affinity endogenous ligand has not been identified. Given the lack of an endogenous ligand having high affinity for PPARγ, and the potential problems associated with artificial PPARγ ligands, the need exists for isolation and characterization of endogenous ligand having high affinity, similar to that found for other nuclear hormone receptors. Furthermore, given the role that PPARγ plays in inflammation and in certain cancers, an endogenous ligand, having high affinity would be and important target for therapy and screening.

The present invention is drawn to an endogenous, neutral lipophilic PPARγ ligand and to methods of isolating PPARγ ligand. In one embodiment, the method of isolation comprises stimulating cells to produce the PPARγ ligand. The cells are present in culture medium, and are stimulated under conditions such that PPARγ ligand is secreted into the culture medium. The culture medium is harvested at about 48 hours after induction of differentiation. Neutral lipophilic compounds are isolated from the harvested culture medium, thereby isolating PPARγ ligand.

In a more particular embodiment, the method of isolating PPARγ ligand comprises inducing disaggregated fibroblast-like cells, e.g., 3T3-L1 cells. The 3T3-L1 cells are contacted with an inducer, e.g., a cAMP inducer, or an inducer that causes the cells to differentiate into adipocytes. In one embodiment, the inducer that causes differentiation into adipocytes comprises a mixture of methyl iso-butyl xanthine at a concentration of about 0.05 to about 5.0 mM, dexamethasone at a concentration of about 0.04 μg/ml to about 4.0 μg/ml and insulin at a concentration of about 0.5 μg/ml to about 50 μg/ml, such that PPARγ ligand is secreted into the culture medium. The culture medium is harvested at about 48 hours after induction of differentiation.

Lipophilic compounds are isolated from the culture medium by extracting the harvested medium into an organic solvent. For example, the organic solvent can comprise a mixture of chloroform and methanol at a ratio of about 2 parts to about 1 part by volume. The organic phase of the extraction step can be further fractionated, for example, by chromatographic methods. In one embodiment, the organic phase is then loaded onto a chromatographic matrix, suitable for the separation of different classes of lipophilic compounds. In a particular embodiment, the organic phase is loaded onto an aminopropyl column.

The PPARγ ligand is eluted with a suitable organic solvent. In one embodiment, the PPARγ ligand is eluted with a mixture of chloroform and isopropanol at a ratio of about 2 parts to about 1 part by volume. This step can optionally be repeated, e.g., the eluted fraction can then be loaded onto a second suitable chromatographic matrix, such as an aminopropyl column, and eluted from the second column with a suitable organic solvent. In one embodiment, the PPARγ ligand is eluted from the second column with a mixture of chloroform and methanol at a ratio of about 2 parts to about one part by volume to obtain a lipophilic eluant which comprises the PPARγ ligand. Using the assay described herein (or other suitable assays), the activity of the PPARγ ligand can be determined at each step of the isolation procedure. In one embodiment, for assessing the activity, the organic fractions containing PPARγ ligand activity are dried and the dried material is resuspended in a suitable aqueous medium for ligand activity measurement. The PPARγ ligand eluted from the column can be further processed (or purified) to obtain enriched fractions of ligand.

The present invention is also drawn to a neutral lipophilic composition comprising PPARγ ligand and to the isolated PPARγ ligand. In one embodiment, the PPARγ ligand is prepared by the method of the present invention.

The present invention is also drawn to a method of increasing PPARγ activity in cells of interest, comprising contacting the cells of interest with isolated PPARγ ligand. In one embodiment, the PPARγ ligand is prepared by a method of the present invention.

The present invention is also drawn to a method of decreasing insulin resistance in an individual. The method comprises administering isolated PPARγ ligand to the individual. In one embodiment, the PPARγ ligand is prepared by the method of the present invention.

The present invention also includes a method for assessing the level of the PPARγ ligand described herein in an individual. The method comprises obtaining a PPARγ ligand-containing sample from the individual and determining the level of PPARγ ligand in said sample in comparison to a control sample, thereby assessing the level of PPARγ ligand in the individual.

The isolation of a novel, endogenous, lipophilic PPARγ ligand that is not a prostaglandin or fatty acid such as linoleic acid was unexpected. It was generally believed that the endogenous ligand was 15-deoxyΔ^{12,14}PG J2. Fatty acids such as linoleic acid and derivatives of linoleic acid were also thought to bind and activate PPARγ. However, as demonstrated herein for the first time, endogenous PPARγ ligand is produced, for example, by pre-adipocytes that have been induced to differentiate into adipocytes. Furthermore, maximal production of the endogenous ligand occurs well before the pre-adipocyte becomes a mature adipocyte.

It was unexpected that the optimal time for harvesting the conditioned medium would be 48 hours after stimulation of the cells. It was traditionally thought that the PPARγ ligand would be expressed later in adipocyte differentiation because the ability of synthetic PPARγ agonists to decrease insulin resistance is very pronounced once an individual has a significant level of adipose tissue.

Surprisingly, the endogenous ligand is not a prostaglandin, nor linoleic acid or derivatives thereof. Rather, purification and enzymatic hydrolysis data reasonably suggests that the ligand is a neutral lipophilic molecule, e.g., a monoglyceride.

The isolated PPARγ ligand of the present invention allows the study of the PPARγ pathway in response to endogenous ligand. As a result of the present invention, the production and regulation of endogenous PPARγ ligand can be examined. The present invention also provides a novel compound for use in decreasing insulin resistance in an individual by binding to and activating PPARγ. Furthermore, the present invention provides an endogenous PPARγ ligand for use in anti-inflammatory and anti-cancer therapy.

### BRIEF DESCRIPTION OF THE DRAWINGS

FIG. 1A is a schematic diagram of the PPARγ ligand activity monitoring assay system.

FIG. 1B is a graph showing the requirement for both PPARγ-LBD and the UAS element for induction in the ligand monitoring system in CV-1 cells.

FIG. 1C is a graph showing the time course of induction of the ligand monitoring system in CV-1 cells.

FIG. 2A is a photomicrograph of 3T3-L1 cells and two lines of 3T3-L1 cells that were stably transfected with the ligand monitoring system taken after the cells had been fully differentiated into adipocytes and stained with Oil Red to visualize lipid content.

FIG. 2B is a Western blot showing levels of PPARγ and C/EBPα expression in 3T3-L1, 5B2 and 5B3 cells at the indicated times after induction of adipogensis.

FIG. 2C is a dose response of 3T3-L1 and 5B2 cells to insulin after full differentiation into adipocytes and serum starvation for 30 minutes.

FIG. 2D is a graph showing the fold increase in β-gal activity in 5B2 and 5B3 cells in response to TZD treatment.

FIG. 3A is a graph showing fold increase in β-gal activity in 5B2 and 5B3 cells at the indicated times after induction of differentiation.

FIG. 3B is a graph showing fold increase in β-gal activity in CV-1 cells transfected with the ligand monitoring system, after treatment of the cells with conditioned medium (CM) or cell extract obtained from 3T3-L1 cells at the indicated time after induction of differentiation into adipocytes.

FIG. 4A is a graph showing the level of β-gal activity in 5B2 and 5B3 cells treated with the indicated compounds.

FIG. 4B is a graph showing β-gal activity in CV-1 reporter cells treated with conditioned media obtained from 3T3-L1 cells treated with the indicated compounds.

FIG. 4C is a graph showing β-gal activity in 5B2 cells stimulated the indicated compounds.

FIG. 4D is a graph showing β-gal activity over time of 5B2 cells treated with the indicated compounds.

FIG. 5 is a graph showing β-gal activity in CV-1 cells transfected with the indicated constructs and incubated with the indicated compound.

FIG. 6A are schematic diagrams of the NBD1-VP and NBD1-DVP constructs.

FIG. 6B is a graph showing β-gal activity in CV-1 cells transfected with the indicated constructs and treated with the indicated concentration of conditioned media.

FIG. 7A show the structure of PD068235.

FIG. 7B is a graph showing the ligand-binding characteristics of PD068235.

FIG. 7C is a graph showing the effect of PD068235 on conditioned media activation of β-gal activity in CV-1 cells transfected with the reporter construction in the presence and absence of NBD1-VP16.

FIG. 8A is a schematic diagram of the cleavage sites of phospholipase A₂, phospholipase C, pancreatic lipase and methanolic base on phosphoglyceride and triglyceride.

FIG. 8B is a schematic diagram showing the digestion of phosphoglyceride with phospholipase A₂.

FIG. 8C is a schematic diagram showing the digestion of phosphoglyceride with phospholipase C.

FIG. 8D is a schematic diagram showing the digestion of triglyceride with pancreatic lipase.

FIG. 8E is a schematic diagram showing the digestion of phosphoglyceride and triglyceride with methanolic base.

FIG. 9 is a flow chart of the purification procedure for PPARγ ligand.

FIG. 10 is a graph showing the effect of COX and LOX inhibitors on the production of PPARγ ligand.

### DETAILED DESCRIPTION OF THE INVENTION

The present invention is drawn toward novel endogenous PPARγ ligand, the isolation of the ligand and the use of the isolated ligand to stimulate PPARγ activity in cells of interest. The invention is also drawn to diagnostic methods to detect the level of the ligand in a sample of interest. As used herein, PPARγ includes splice variants PPARγ1, PPARγ2, and PPARγ3.

The present invention is drawn to a method of isolating PPARγ ligand. In one embodiment, the method comprises stimulating cells to produce PPARγ ligand wherein at least a portion of the PPARγ ligand is secreted into the culture medium. The culture medium is then harvested. In one embodiment, the culture medium is harvested at about 48 hours after stimulation. Neutral lipophilic compounds are obtained from the harvested culture medium, thereby isolating PPARγ ligand.

The cells used in the method of isolating PPARγ ligand can comprise any cell type capable of producing the PPARγ ligand of the present invention or capable of being induced to produce the PPARγ ligand of the present invention. The cells can comprise cultured cells. Cultured cells can be cell lines, primary culture or tissue sections. The tissue sections can be perfused. The cultured cells can be derived from any suitable organism. Suitable organisms include rodents such as mouse or hamster, livestock such as pigs, goats or cows, non-human primates and humans. In one embodiment, the cells comprise pre-adipocytes. As used herein, pre-adipocytes are cells that can be induced to differentiate into adipocytes. As used herein, differentiation into adipocytes includes alteration of cells such that the altered cells contain lipid droplets.

Cell lines suitable for use in the method of producing PPARγ ligand include cell lines derived from disaggregated fibroblast-like cells (e.g. 3T3-L1 cells or 3T3-F442A cells) or cells derived from the stroma of epididymal fat pads of adult ob/ob mice (e.g. Ob1771 cells). The primary culture can be derived from any tissue that produces PPARγ ligand, e.g. primary adipocytes or adipose tissue.

In one embodiment, of the method of isolating PPARγ ligand, cultured cells are induced to differentiate into adipocytes. The cultured cells are contacted with a composition comprising methyl iso-butyl xanthine at a concentration of about 0.05 to about 5.0 mM, dexamethasone at a concentration of about 0.04 μg/ml to about 4.0 μg/ml and insulin at a concentration of about 0.5 to about 50 μg/ml, under conditions such that PPARγ ligand is produced and at least a portion is secreted into the culture medium. In a more particular embodiment, the cells are contacted with methyl iso-butyl xanthine at a concentration of about 0.5 mM, dexamethasone at a concentration of about 0.4 μg/ml and insulin at a concentration of about 5 μg/ml.

Applicants have also found that stimulation of the cells such that intracellular cAMP levels are elevated is sufficient to induce the production and secretion of the PPARγ ligand. Therefore, to induce the production of PPARγ ligand, the cells can be stimulated with a compound that elevates intracellular cAMP levels, thereby stimulating the cells to produce PPARγ ligand. In one embodiment, the cAMP elevating compound is selected from the group consisting of: methyl iso-butyl xanthine, 8-bromo-cAMP, and Forskolin. The cAMP elevating compounds are present in sufficient levels to cause elevation of cAMP in the cells, for example, methyl iso-butyl xanthine can be used at a concentration of about 0.1 to about 1 mM, 8-bromo-cAMP can be used a concentration of about 0.03 to about 0.3 mM and Forskolin can be used at a concentration of about 2.5 to about 25 μM.

According to the method of the present invention for isolating PPARγ ligand, neutral lipophilic compounds are isolated from the harvested culture medium. For example, in one embodiment, the harvested medium is extracted with solvent comprising an organic phase, such that lipophilic compounds partition into the organic phase, and the non-lipophilic compounds do not. Methods of extracting lipophilic compounds from aqueous solutions are well known in the art. In one embodiment, the extraction solvent comprises a mixture of chloroform and methanol. In a more particular embodiment, the chloroform/methanol mixture comprises about 2 parts of chloroform and about 1 part or methanol by volume. One of ordinary skill in the art can vary the ratio of chloroform to methanol and the total amount of extraction solvent to extract the lipophilic compounds as described herein using no more than routine experimentation. Furthermore, other extraction solvents may be used, so long as PPARγ ligand activity can be detected in the resulting ligand-containing fractions.

The organic phase of the chloroform/methanol extraction step is loaded onto a chromatographic column, wherein the column comprises a matrix suitable for separating classes of lipophilic compounds and under conditions such that the PPARγ ligand activity is bound to the column. In one embodiment, an aminopropyl column is used. The bound lipophilic compounds containing the PPARγ ligand activity are eluted with a suitable solvent. The elution solvent can comprise a mixture of chloroform and isopropanol. In one embodiment, the elution solvent comprises about 2 parts chloroform to about 1 part isopropanol by volume.

The PPARγ ligand activity containing fractions, eluted from the column, can be further fractionated, for example, using a second column chromatography step. In one embodiment, a second aminopropyl column is used. The bound lipophilic compounds containing the PPARγ ligand activity are eluted with a suitable solvent. The elution solvent can comprises a mixture of chloroform and methanol. In one embodiment, the elution solvent comprises about 2 parts chloroform to about 1 part methanol by volume. In another embodiment, the elution solvent comprises ethyl acetate and acetone. The ethyl acetate/acetone solvent can be, for example, at a ratio of about one part ethyl acetate to about one part acetone.

The PPARγ ligand containing fractions can be further purified by subjecting the eluted material to HPLC using a reversed phase column. The use of the reversed phase column can be as described, for example by Lopez et al., Journal of Chromatography B, 760:97 (2001), the teachings of which are incorporated herein by reference in their entirety. In one embodiment, the column is a C18 column. In one embodiment, the mobile phase comprises a solvent suitable to separate the PPARγ ligand from other non-ligand lipophilic components of the extract. The mobile phase can comprise acetonitrile and acidified water. In a more particular embodiment, the mobile phase comprises 98.6% acetonitrile and 1.4% acid water. The acid water can comprise, for example, 0.035% formic acid, pH 2.62.

In one embodiment, the dried fraction is resuspended in 250 μl acetonitrile. The column can be of experimental or preparatory scale. Where the column is a 3.0×150 mm C18 column, and is run using isocratic elution with a running time of 10 min. and a flow rate of 0.7 ml/min., the PPARγ elutes from the HPLC column between about 2 min. 45 sec. and about 3 min. 15 sec. In a more particular embodiment, the PPARγ ligand elutes from the column between about 2 min. 45 sec. and 3 min. The size of the HPLC column, elution profile and flow rate can be changed and the effect on elution time determined using the PPARγ ligand detection methods described herein.

The present invention is also drawn to isolated and/or purified endogenous PPARγ ligand and to compositions comprising the PPARγ ligand. In one embodiment, the invention is drawn to the PPARγ ligand isolated by the method described herein. In one embodiment, the PPARγ ligand comprises a non-polar and neutral lipid. In still a more particular embodiment, the PPARγ ligand comprises a monoacylglyceride, also referred to herein as a monoglyceride. In one embodiment, the PPARγ ligand comprises a sn-2-monoacylglyceride. The PPARγ ligand of the present invention is characterized as being a neutral, oxidation sensitive, lipophilic compound that is not a triglyceride, diglyceride, or sn-1-(or alpha-) monoglyceride.

Based on the physical parameters and activity of PPARγ ligand as described herein, one of ordinary skill in the art can synthesize and isolate the PPARγ ligand using standard chemical or enzymatic techniques and using the PPARγ activity monitoring system as described herein to assess the activity of the ligand and follow the PPARγ ligand through the purification steps.

Methods for producing monoglycerides are also described in U.S. Pat. No. 5,316,927 to Zaks, et al., the teachings of which are incorporated herein by reference. The methods include glycerolysis of fats. The fatty acid groups of triglycerides are transferred to the hydroxyl groups of glycerol and the monoglycerides are isolated by distillation. Another method involves enzymatic transformation, including the esterification of glycerol with fatty acid, the glycerolysis of triglycerides and the partial hydrolysis of triglycerides. A particular method described in U.S. Pat. No. 5,316,927 is lipase-catalyzed transesterification of triglycerides in an alcohol medium. Methods for producing monoglycerides are also described in U.S. Pat. No. 5,153,126 to Schroder et al., the teachings of which are incorporated herein by reference.

As used herein the terms “isolating” and “isolated” refers to PPARγ ligand where at least a portion of non-PPARγ ligand has been removed compared to PPARγ ligand present in the starting material, e.g., conditioned medium, lysed cells or synthesis product. The non-PPARγ ligand can include proteins, nucleic acids and lipophilic compounds that do not have PPARγ ligand activity. In one embodiment, at least 90% of proteins found in PPARγ ligand containing starting material has been removed. In another embodiment, at least 90% of the non-PPARγ ligand lipophilic compounds found in PPARγ ligand containing conditioned medium or lysed cells have been removed. In a more particular embodiment, at least 95% or at least 99% of the proteins and non-PPARγ ligand lipophilic compounds have been removed. The present invention also includes synthetically produced PPARγ ligand having the structural properties and PPARγ activity as described herein. Synthetically produced PPARγ ligand can include other compounds, including protein or other hydrophilic compounds and still be considered isolated. It is understood that non-PPARγ ligand can be present in the isolated or synthetically produced PPARγ ligand of the present invention, without affecting the PPARγ ligand activity.

The present invention is also drawn to a method of increasing PPARγ activity in cells of interest. The method comprises contacting the cells with the isolated PPARγ ligand described herein, under conditions such that PPARγ activity in the cells of interest is increased.

As used herein, PPARγ activity can include intracellular and extracellular changes. For example, PPARγ activity can include one or more of the following: decreased insulin resistance, apoptosis, induction of fat cell differentiation, induction of PPARγ-containing cells to differentiate at least partially into adipocytes, binding of PPARγ to PPARγ responsive DNA elements, and activation of PPARγ responsive genes. Activities can further include therapeutic uses for certain tumors such as liposarcomal or in colon or breast cancers, or control of cardiovascular diseases and hypertension. These activities can be measured using standard protocols known in the art. PPARγ responsive DNA elements include, for example, the DR-1 element. Binding of PPARγ to the PPARγ responsive elements can be measured by any suitable DNA binding method known in the art, e.g., gel shift assay. PPARγ responsive genes include, for example, adipsin, aP2, lipoprotein lipase and PPARγ. Increases in gene expression can be measured by standard techniques in the art, such as by probing cellular RNA with specific detectable nucleic acid probes, for example as described in Tontonoz et al., Cell 79:1147-1156 (1994), the teachings of which are incorporated herein by reference in their entirety. As used herein, an increase in PPARγ activity includes an increase of at least 5% of the particular PPARγ activity in question. In more particular embodiments, the increase in PPARγ activity includes an increase of at least 10, 25, 50, 75, 90, 95 and 99% increase in the particular PPARγ activity in question. As used herein, an increase in PPARγ activity includes transient increases.

The cells of interest in the method of increasing PPARγ activity can be any cells that express or can be induced to express PPARγ. The cells of interest include normal and neoplastic (e.g., cancer) cells. The cells of interest include pre-adipocytes, adipocytes, liposarcoma cells, cells of the large and small intestine, e.g. epithelial cells, hematopoietic cells, monocytes, macrophages, kidney cells, liver cells, breast epithelial cells, including breast cancer cells and muscle cells.

The present invention is also drawn to a method of decreasing insulin resistance in an individual. Insulin resistance is characterized, for example, by increased glucose concentration in the blood, increased insulin concentration in the blood, decreased ability to metabolize glucose in reponse to insulin, or a combination of any of the above. The method comprises administering isolated PPARγ ligand to the individual. In one embodiment, the ligand is isolated by inducing cultured cells to differentiate into adipocytes, such that PPARγ ligand is secreted into the culture medium. The culture medium is harvested at about 48 hours after induction of differentiation, and lipophilic compounds are isolated from the harvested culture medium, such that insulin resistance is decreased.

The ligand can be administered orally, mucosally, nasally, by inhalation, by suppository, topically and by injection. As used herein, injection includes intraperitoneally, intravenous, intramuscular, subcutaneous and into adipose tissue. The lipophilic ligand of the present invention is expected to be readily absorbed by the cells of interest. Pharmacological excipients can be added to the ligand of the present invention to facilitate ligand reaching the cell of interest. For example, the ligand can be mixed with physiologically acceptable excipients and administered as described in U.S. Pat. No. 6,294,580 to Willson et al., the teachings of which are incorporated herein by reference.

The amount of ligand to be administered can be determined through routine experimentation to give the desired effects. In one embodiment, the endogenous ligand of the present invention has an affinity for PPARγ similar to or greater than, that found for the thiazolidinedione class of synthetic PPARγ agonists (e.g., the tens of nanomolar range). In one embodiment, the isolated endogenous ligand of the present invention can be administered to an individual at about 0.05 mg/kg/day to about 50 mg/kg/day.

The present invention is also drawn to a method for assessing the level of PPARγ ligand in an individual. The method comprises obtaining a PPARγ ligand-containing sample from the individual. The level of PPARγ ligand in the sample is measured and compared to the level of activity in a control sample.

In the method for assessing the level of PPARγ ligand, the individual can be any mammal, as described above. In one embodiment, the sample is from any tissue or bodily fluid that contains the PPARγ ligand of the present invention. In a more particular embodiment, the sample is selected from the group consisting of: blood and adipose tissue. As a control for the level of PPARγ ligand, a control sample can be prepared from a normal individual or a known quantity of PPARγ ligand can be used.

The level of PPARγ ligand is then determined. In one embodiment, the level of PPARγ activity is measured by exposing cultured PPARγ ligand monitoring cells to the sample or control. The monitoring cells can be any cell type as described herein, capable of responding to PPARγ ligand. In one embodiment, the monitoring cells are pre-adipocytes and the read-out is differentiation into adipocytes. In another embodiment, the monitoring cells are cells that have been transformed with a PP ARγ activity reporter system as described herein and the read out is β-gal or luciferase activity.

Thus, as a result of the work described herein, a novel, endogenous PPARγ ligand is provided. The PPARγ ligand of the present invention can be used to increase PPARγ activity in cells of interest and more particularly, can be used to decrease insulin resistance in an individual. As a result of the present invention, individuals can also be tested and monitored for the level of PPARγ ligand.

### EXEMPLIFICATION

### Example 1

### PPARγ Ligand Monitoring System; Construction and Initial Testing

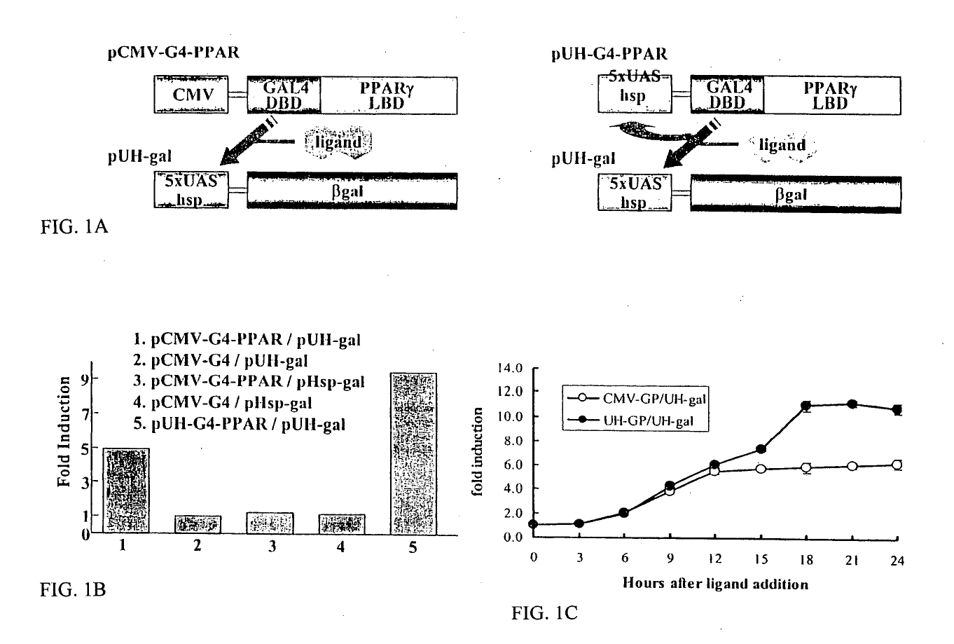

A PPARγ ligand monitoring system was constructed following the method of Alexander Mata de Urquiza et al. (Proc. Natl. Acad. Sci. U.S.A., 96:13270-13275 (1999). Effector constructs were made by fusing the nucleic acid sequence encoding the ligand-binding domain of PPARγ (PPARγ-LBD), to nucleic acid encoding the DNA-binding domain of the yeast Gal4 transcription factor (GAL4-DBD). Briefly, a fragment comprising nucleotides 487-1428 of the PPARγ gene as recorded in GenBank, accession number U01664 were cloned downstream of Gal4 DNA binding domain (encoding amino acids 1-147), in frame. For one effector construct, the GAL4-PPARγ fusion was under the control of the cytomegalovirus (CMV) promoter, generating the construct pCMV-G4-PPARγ. For another effector construct, the GAL4-PPARγ fusion was under the control of the Gal4-specific binding site (5×UAS) linked to the hsp68 minimum promoter (pUH-G4-PPARγ). A reporter construct pUH-gal was made containing a bacterial β-galactosidase gene driven by 5×UAS linked to the hsp68 minimum promoter. As shown in FIG. 1A, the pCMV-G4-PPARγ effector construct is expected to bind to and activate the 5×UAShsp promoter of the reporter construct upon interaction with PPARγ ligand, while the pUH-G4-PPARγ effector construct is expected to activate both the pUH-G4-PPARγ and the reporter construct by binding to the 5×UAShsp promoters present in each construct. Therefore, the pUH-G4-PPARγ system is expected to result in a positive feedback in signal upon interaction of PPARγ with ligand.

The effector and reporter constructs were transfected into either CV-1 cells (monkey kidney cells) or 3T3-L1 cells (murine fibroblast cells capable of being differentiated into adipocytes). Cells were grown according to standard protocols for these cells. For example, 3T3-L1 cells were grown and maintained as fibroblasts in DMEM/high glucose, containing 10% calf serum in a 10% CO₂ humidified environment at 37° C. The cells were maintained at a subconfluent level so as to not prematurely arrest cell growth and induce differentiation. The cells were typically split 1:5 or 1:3 every 3 to 4 days. Cells were transfected using Lipofectamine™ 2000 Reagent (Life Technologies™, Carlsbad, Calif.) according to the manufacturers instructions. Cells were plated in 24 well dishes for the assay.

As shown in FIG. 1B, induction of β-galactosidase activity was detected when pCMV-G4-PPARγ or pUH-G4-PPARγ are co-transfected with pUH-gal to CV-1 cells in the presence of synthetic ligand (1 μM Troglitazone). β-gal activity was measured by rinsing the cells in the 24 well dishes with 1.5 ml/well PBS one time. The washed cells were lysed with 200 μl lysis buffer (25 mM glycylglycine, 15 mM MgSO4, 4 mM EGTA, 1 mM DTT, 1% Triton) per well. Ten μl of the lysate were mixed with 150 μl of 100×diluted galacton (Tropix, Bedford, Mass.), and incubated at room temperature for 30 min. The β-gal activity was measured for 10 sec. in a Luminometer (LB9507, EG&G, Bad Wildbad, Germany). As expected, higher induction was observed when pUH-G4-PPARγ was transfected, compared with pCMV-G4-PPARγ. The induction of β-gal activity required the presence of both PPARγ-LBD and UAS.

As shown in FIG. 1C, no obvious delay of the start of induction of β-gal activity was observed using the feedback-inducible system compared with the traditional reporter system.

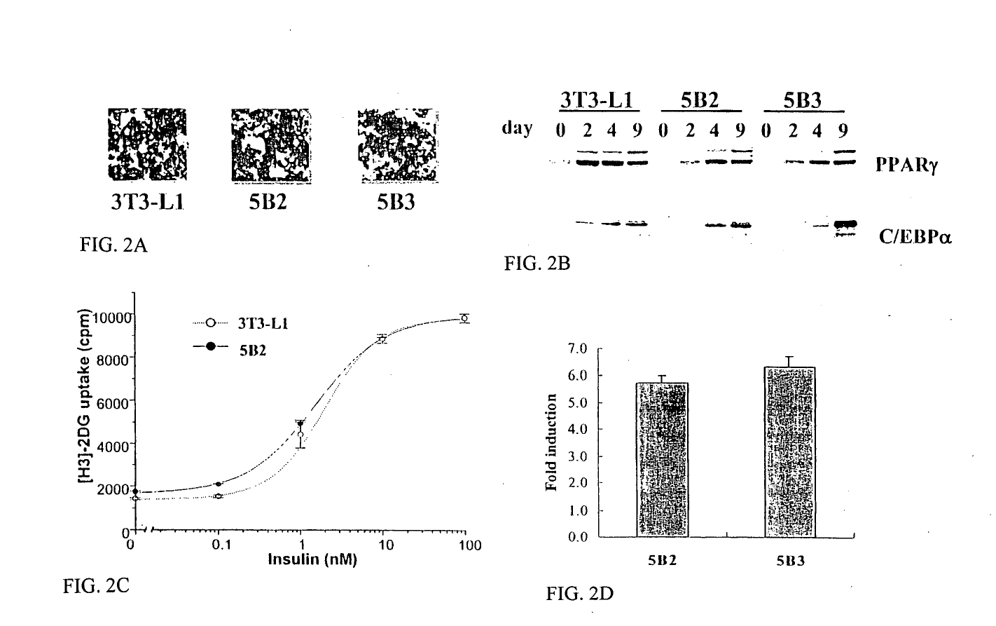

3T3-L1 cells were transfected with the feedback monitoring system as described above and two stably transfected cell lines, 5B2 and 5B3 were isolated. 5B2 and 5B3 were then grown to confluence and induced to differentiate into adipocytes following standard differentiation methods (See Green and Kehinde, Cell, 1:113-116 (1974); Rise et al., J. Biol. Chem., 267:10163-10167 (1992); and Herreos and Birnbaum, J. Biol. Chem., 264:19994-19999 (1989), the teachings of which are incorporated herein by reference). Briefly, the cells were plated in 24 well dishes at a density of 3.3×10³ cells per well. The cells were maintained until they reached complete confluence (day 0). Differentiation was induced in day 0 cells by adding differentiation media, MIX-Diff for two days. MIX-Diff media was prepared by adding 10% fetal bovine serum, 5 μg/ml insulin, 0.4 μg/ml dexamethasone (Dex, stock 4 mg/ml in EtOH and stored at −20° C.) 0.5 mM methyl iso-butyl xanthine (MIX, add solid MIX in PBS to give final 5 mM and heat to almost boiling to dissolve, dispense and store at −20° C.) to DMEM/high glucose and filter sterilizing. MIX-Diff was used within 4 weeks of preparation. On day2, DMEM/high glucose containing 10% FBS and 5 μg/ml insulin was added. On day 4, DMEM/high glucose containing 10% FBS was added. The cells were typically fully differentiated by day 8 to day 10. 3T3-L1 cells were also induced as a control. At day nine of differentiation, the cells were stained with Oil Red to visualize the lipid deposits. As shown in FIG. 2A, 5B2 and 5B3 showed normal adipogenic differentiation.

3T3-L1, 5B2 and 5B3 cells were also induced to differentiate into adipocytes as described above and at the indicated times following induction of differentiation, total protein extracts were analyzed for expression of PPARγ and C/EBPα by Western blot. As shown in FIG. 2B, PPARγ1 (lower band) and PPARγ2 (upper band) expression increased to maximal levels by day 2 after induction and declined thereafter until the last measurement on day 9, while C/EBPα also increase after induction but continued to rise until day 9. For 5B2 and 5B3, PPARγ expression increased upon induction and continued to increase until days 4 and 9, while C/EPBα expression increased by day 4 and continued to increase by the last measurement, day 9.

In a separate experiment, fully differentiated 3T3-L1 or 5B2 cells from day 9 after induction of differentiation were serum-starved for 3 hours and then stimulated for 30 minutes with insulin at the indicated concentrations. Cells were washed one time with DMEM without serum and without insulin (DMEM0). The washed cells were incubated for 3 h in 500 μl DMEM0. After incubation, the cells were washed one time with 500 μl/well of glucose-free MEM. Three hundred μl/well glucose-free MEM +/−100 nM insulin was added to each well and incubated for 30 min. Stock solution of [H³]-2-deoxy-D-glucose (DOG) was prepared by combining 960 μl glucose-free MEM, 30 DOG and 10 μl [H³]DOG. Ten μl of the [H³]DOG mix was added to each well and the wells were incubated for 10 min. The cells were then transferred to ice and 500 μl cold PBS+phloretin was added. Phloretin was prepared by dissolving 8.2 mg phloretin in EtOH and then bringing the volume up to 100 ml with PBS; the mixture was protected from light. Cells were then washed two times with cold PBS. The cells were then lysed in 400 μl 1N NaOH for 30-60 min and the lysate was transferred to scintillation vials together with 50 μl concentrated HCl. H³ was measured. As shown in FIG. 2C, cells stably transfected with the ligand monitoring system responded similarly to parental 3T3-L1 cells, indicating that the monitoring system did not disrupt PPARγ function in the transfected cells.

The response of β-gal activity of the stable cell lines to TZD was examined by incubating the growing pre-adipocyte cells with or without 2 μM Troglitazone for 16 hours. As shown in FIG. 2D, both cell lines showed at least 5 fold induction in β-gal activity in response to TZD treatment.

### Example 2

### Time Course of PPARγ Ligand Production During Adipogensis

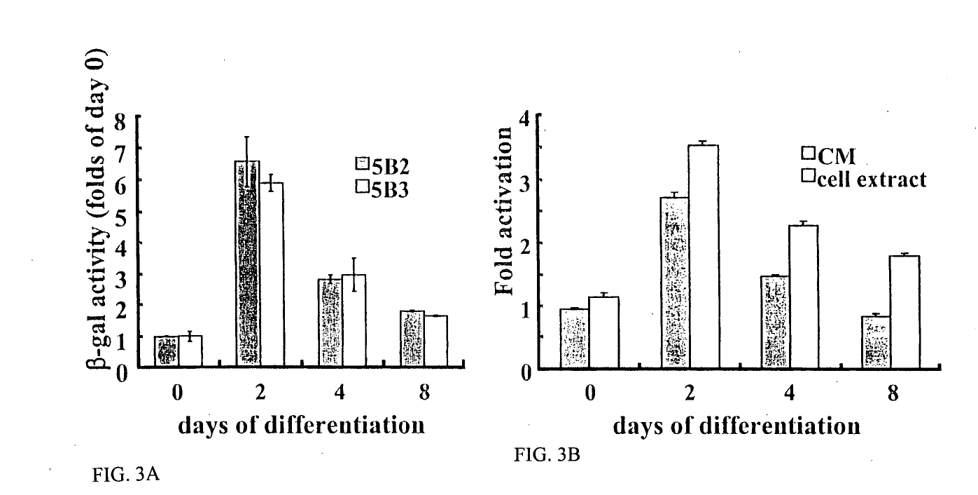

The cell lines 5B2 and 5B3 were induced to differentiate as described above. At the indicated times following induction of differentiation, the cells were lysed and β-gal activity measured as described above. As shown in FIG. 3A, both 5B2 and 5B3 showed maximal β-gal activity at day 2 after induction, indicating that the cells produced maximal PPARγ ligand early during the differentiation into adipocytes.

### Example 3

### PPARγ Ligand is Secreted by Induced Cells

To determine whether the ligand was being secreted, PPARγ ligand activity in conditioned media and extracts of differentiating 3T3-L1 was measured. At the indicated times following induction of differentiation, the conditioned media was collected, and the cells were extracted with ethyl acetate and acetone. Cell extracts were obtained from cells grown and induced to differentiate in 10 cm dishes. Briefly, on the indicated day after induction, the dishes were rinsed twice with 15 ml PBS and scraped into 12 ml PBS. The extraction was performed by adding 600 μl 2M HCl, 12 ml ethyl acetate and 12 ml acetone to the cell suspension. The mixture was shaken for 5 min and centrifuged at 100×g for 5 min. The upper phase, containing molecules soluble in organic solvents was transferred to a new tube and evaporated in a SpeedVac (Savant). The dried samples were prepared for ligand activity assay by dissolving in PBS, the same volume as the original volume from the starting dishes.

The conditioned media and cell extracts were used to treat CV-1 cells, which had been transfected with the monitoring constructs one day before and were grown in 24-well plates with 0.5 ml of culture media per well. β-gal activity was measured after 24 hours of treatment as described above. As shown in FIG. 3B, the PPARγ ligand activity was found in both conditioned media and in cell extracts. The activity dramatically increased at day 2 after the induction, then decreased gradually throughout differentiation thereafter to levels found in pre-adipocytes by day 8.

### Example 4

### Day Two Conditioned Media Promotes Adipogensis of NIH3T3 cells transfected with PPARγ.

NIH-3T3 cells were infected with the empty vector pBabe (Cell, 79:1147-1156 (1994), the teachings of which are incorporated herein by reference) or pBabe encoding PPARγ2. The infected cells were induced to differentiate as described above or were treated with 5 μM Troglitazone, day 2 conditioned media (CM) from differentiating 3T3-L1 cells, extracts of day 2 CM from differentiating 3T3-L1 cells, or day 8 CM from differentiating 3T3-L1 cells. Conditioned media was obtained by harvesting the media from the treated cells at the indicated time. CM was used neat. Extracts were prepared as described in Example 10.

After 8 days, the treated cells were stained with Oil Red to visualize lipid content. The day 2 conditioned media and the extracts promoted the lipid accumulation within the PPARγ2 infected NIH-3T3 cells to a similar extent as TZD treatment. Day 8 conditioned media promoted lipid accumulation to a lesser extent.

### Example 5

### cAMP Induced PPARγ Ligand Production During Adipogensis

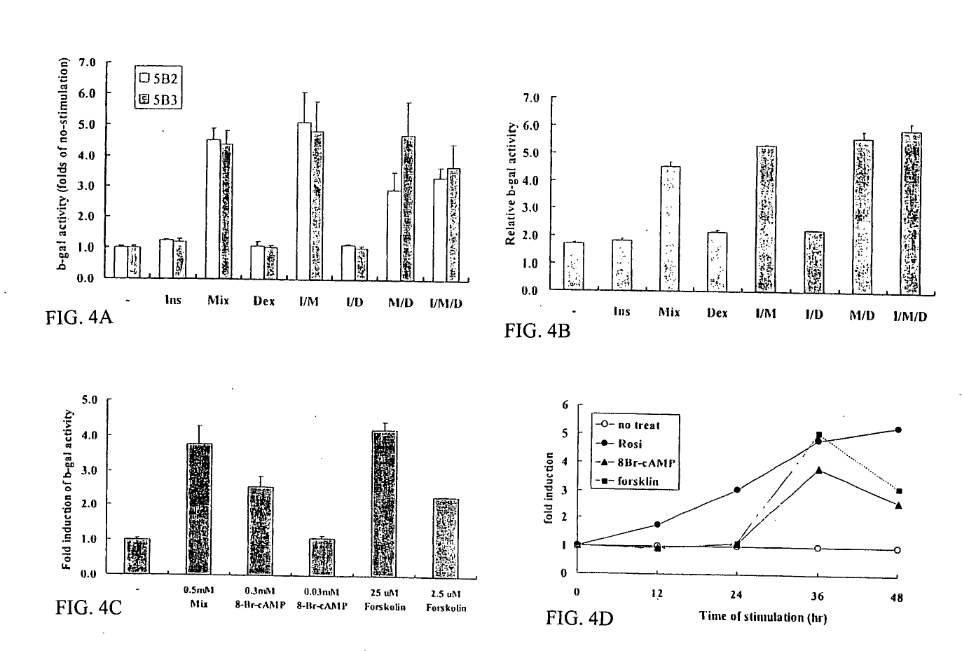

To determine the effect of each component in adipogenic mixture on ligand production, the monitoring cells, 5B2 and 5B3, were stimulated by exposure to different combinations of 5 μg/ml Ins, 0.5 mM Mix and 0.4 μM Dex, and the β-gal activity were measured at day 2 after stimulation. As shown in FIG. 4A, the induction of β-gal activity was dependent on the stimulation by Mix.

The conditioned media from 3T3-L1 cells stimulated by different combinations of Ins, Mix and Dex for 2 days were collected and incubated with CV-1 cells transfected with the ligand monitoring system. As shown in FIG. 4B, the ligand production during adipogensis is also dependent on stimulation by Mix in normal 3T3-L1 cells.

The monitoring cell line 5B2 was stimulated by exposure to Mix, 8-Bromo-cAMP or Forskolin. In FIG. 4C, β-gal activity was measured at day 2. In FIG. 4D β-gal activity was measured at the indicated time and as a control, the cells 5B2 were treated with 0.2 μg/ml Rosi. These data indicated that Mix induced the ligand production of differentiating pre-adipocytes by increasing the intracellular cAMP.

### Example 6

### The Conditioned Media Specifically Induces PPARγ Activity

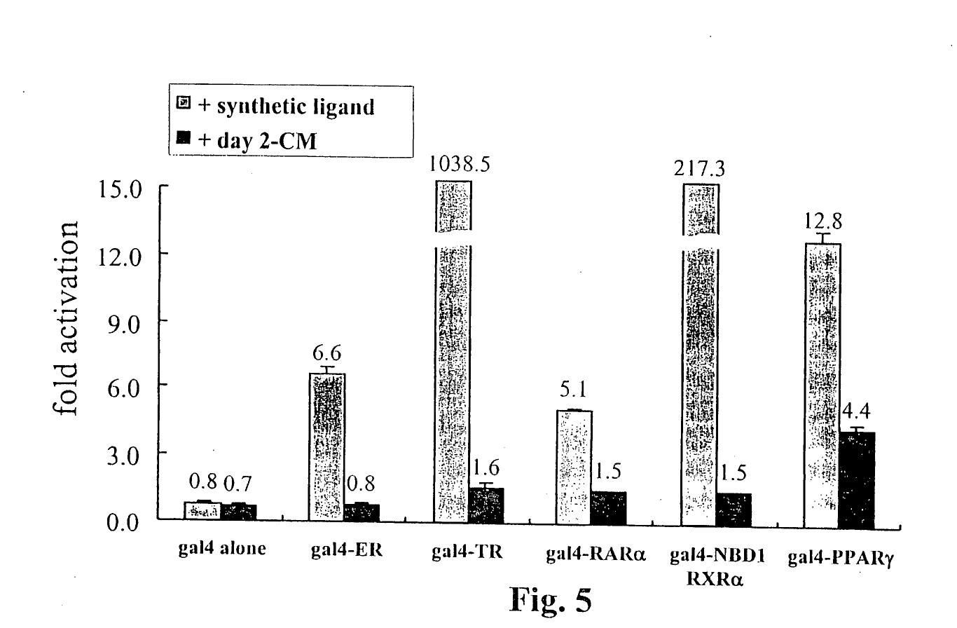

CV-1 cells were transfected as described above with pUH-gal and either expression vectors encoding the Gal4 DNA-binding domain (gal4 alone), or the fusion protein of the Gal4 DNA-binding domain and the ligand binding domain of either estrogen receptor (gal4-ER), thyroid receptor (gal4-TR), RARα (gal4-RARα), PPARγ (gal4-PPARγ) or RXR and fusion protein of Gal4 DNA-binding domain and NBD1 of SRC-1 (gal4-NBD1/RXR). After transfection, the cells were incubated with day 2-CM or synthetic ligands (1 μM Troglitazone for gal4 alone and gal4-PPARγ, 1 μM E2 for gal4-ER, 1 μM T3 for gal4-TR, 1 μM cis-9 RA for gal4-RARα and gal4-NBD1/RXR) for 20 h. β-gal activity was measured as described above. As shown in FIG. 5, the two day conditioned media induced PPARγ activity only in cells transfected with the gal4-PPARγ expression vector. The numbers at the top of each column indicate the fold activation by the treatment.

### Example 7

### PPARγ Coactivator SRC-1 Potentiates Ligand Activity of the Conditioned Media

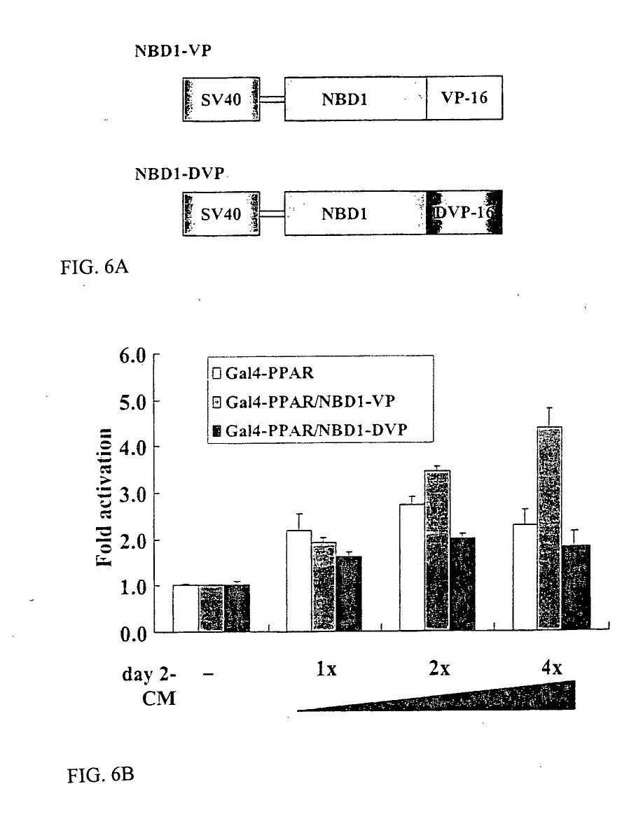

CV-1 cells were transfected as described above with pUH-G4-PPARγ and pUH-gal, with or without nuclear receptor interaction domain NBD1 of SRC-1 fused with VP16 or inactivated VP16. The NBD-1 nuclear receptor binding domain was fused with VP-16 in an expression vector (NBD1-VP) under the control of an SV40 promoter. As a control, NBD-1 was also fused to inactive VP16 in a separate expression vector (NBD1-DVP), also under the control of an SV-40 promoter. The two NBD1 constructs are shown in FIG. 6A. After transfection, cells were incubated in the absence or presence of CM extracts obtained as described above for cell extracts. The CM extracts were concentrated by resuspending the extract in ½ or ¼ of the starting volume of CM to obtain the indicated concentrations. The treated, transfected cells were harvested and β-gal activity measured 48 hrs post-transfection. As shown in FIG. 6B, co-transfection of NBD-1-VP16 increased the reporter activity induced by the conditioned media.

### Example 8

### PPARγ Specific Antagonist PD068235 Blocks the Ligand Activity of Conditioned Media

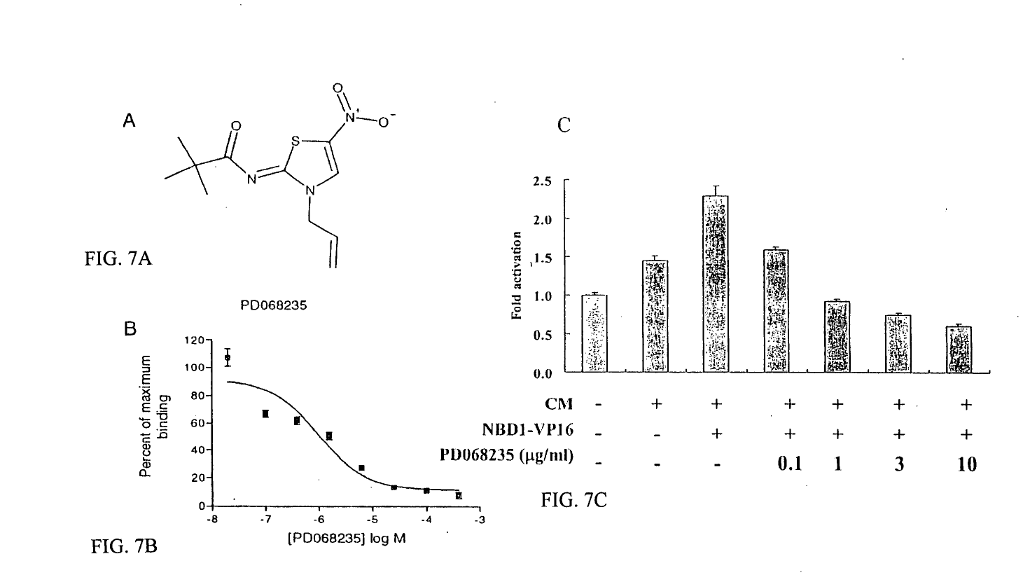

The compound PD068235, shown in FIG. 7A, was reported as a potent full antagonist of PPARγ. It displaces rosiglitazone with a Ki about 1 μM (FIG. 7B). This compound has also been shown to inhibit the interaction of coactivator SRC-1 with synthetic agonist-bound PPARγ and prevent 3T3-L1 adipocyte differentiation (H. Camp et al., Endocrinology, 142:3207-3213 (2001)).

CV-1 cells were transfected with an expression vector encoding full-length PPARγ1 (pCMV-PPARγ) and a reporter plasmid p3DR1-1uc. p3DR1-luc contains a triple copy of PPARγ response element, Direct Repeat 1, linked to the luciferase gene. The cells were transfected with the reporter with or without NBD1-VP16 expression vector. The transfected cells were incubated with or without conditioned media in the absence or presence of various concentrations of PD068235. The cells were washed and lysed as described above for the β-gal assay, except that 50 μl of the lysate were mixed with 100 μl assay buffer and measured for 15 sec. in the Luminometer. Assay buffer consisted of 25 mM glycylglycine, 15 mM MgSO₄, 4 mM EGTA, 1 mM DTT, 15 mM KH₂PO₄, 6 mM ATP and 100 μl of a 0.225 mg/ml luciferin stock solution. As shown in FIG. 7C, the PPARγ specific antagonist showed a dose-dependent inhibition of CM-stimulated PPARγ transcriptional activity.

### Example 9

### PPARγ Ligand Production Is Not Dependent Upon COX or LOX

To test whether PPARγ ligand production was dependent upon cyclooxygenase (COX) or lipooxygenase (LOX), which would be expected if the ligand were a prostaglandin, the effect of COX and LOX inhibitors on PPARγ ligand production during 3T3-L1 differentiation into adipocytes was examined by measuring the β-gal activity in the stable cell line, 5B2.

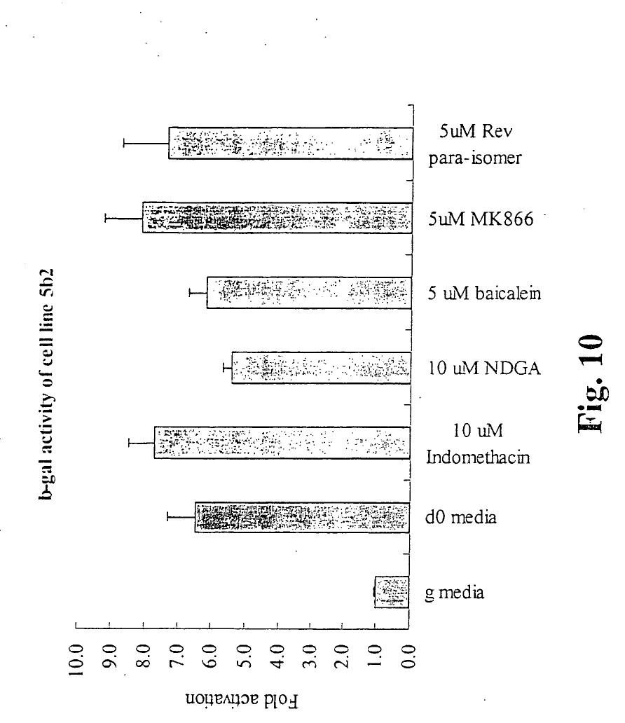

5B2 cells were stimulated to differentiation into adipocytes as described above. The media containing the mixture of insulin, DEX and MIX is called d0 media on FIG. 10. In the experimental samples, d0 media contained the indicated COX or LOX inhibitor. The fold induction of β-gal activity of day 2 cells was compared to cells of day 2 stimulated in the absence of hormones (g media).

As shown in FIG. 10, 10 μM Indomethacin (a non-selective COX inhibitor), 10 μM NDGA (LOX inhibitor), 5 μM Baicalein (12-LOX inhibitor), 5 μM MK866 (5-LOX activation inhibitor) and 5 μM REV 5901-para-isomer (5-LOX activation inhibitor) had no significant effect on the β-gal activity produced by the stimulated 5B2 cells, indicating that the products of COX and LOX reactions are not the ligand itself and not involved in ligand production during adipogensis. This indicates that PPARγ ligand is not a prostaglandin.

### Example 10

### Purification of the PPARγ Ligand

Molecules with ligand activity were isolated from conditioned medium by stepwise fractionation using preparative methods. Activity in the resulting fractions was tested. Analytical methods were performed to confirm the effectiveness of isolation.

1. Preparative methods

Organic Extraction

A general extraction of lipid compounds from conditioned medium was performed following a variation of Folch's method (Folch et al., J Biol Chem, 226: 497 (1957)), by addition of 6 volumes of chloroform:methanol (2:1, v/v) to the conditioned medium. Glass centrifuge tubes containing samples were vortexed briefly and centrifuged at 800×g for 5 minutes to separate two phases. The upper phase contained water soluble compounds, and the lower phase contained molecules soluble in organic solvents. Proteins partitioned in the interphase. The lower phase was transferred to a new tube, evaporated under a nitrogen stream and prepared for ligand activity assay. The samples were concentrated by resuspending the dried material in a smaller volume of culture media. Typically, the dried material was resuspended in 1/12 of the starting volume. Where the cells were grown in 24 well plates, each well contained 0.5 ml of media and three wells were combined per assay. The evaporation step was performed at room temperature to avoid oxidation. Samples were kept on ice for the rest of the process.
Solid Phase Extraction

The lower phase obtained from the organic extraction was subjected to solid phase chromatography extraction by aminopropyl-bonded silica gel columns (Supelclean LC-NH2-SPE, Supelco, Bellefonte, Pa.), following the method described by Kaluzny et al. (J Lipid Research, 26:135 (1985)) and modified by Alvarez and Touchstone (J Chromatography B, 577:142 (1992)). Columns were placed onto a Vac Elut-20 vacuum instrument (Varian, Palo Alto, Calif.) and conditioned with 2 ml of hexane at an approximate flow rate of 2 m/min.

Evaporated extracts (from 1 ml of CM) were resuspended in 200 μl of chloroform and sonicated in a Branson sonication bath (Branson Ultrasonics, Danbury, Conn.) at room temperature for 3×5 sec and loaded onto columns (equivalent of 4 ml of CM per column), allowing the solvent to reach the top of the column by gravity.

Samples were eluted sequentially with 4 ml of four different solvents (flow rate 2 ml/min) and the corresponding fractions collected separately. Fraction 1 was eluted with chloroform:isopropanol (2:1, v/v), and contained non-polar lipids (cholesterol, cholesterol esters, and glycerides). Fraction 2 was eluted with ethyl ether:acetic acid (98:2, v/v) and contained free fatty acids. Fraction 3 was eluted with methanol and contained neutral polar lipids, including phosphatidylethanolamine, phosphatidylcholine, sphingomyelin and neutral glycolipids. Fraction 4 was eluted with chloroform:methanol:0.8 M sodium acetate (60:30:4.5, v/v/v) and contained polar acidic lipids, including phosphatidylglycerol, cardiolipin, phosphatidylinositol, phosphatidylserine and acidic glycosphingolipids.

Evaporated Fraction 1 extracts were resuspended in 200 μl of hexane and subjected to a second solid phase extraction process, using five different solvents in the same conditions described in the previous paragraph. Fraction 1-1 was eluted with hexane, and contained cholesterol esters. Fraction 1-2 was eluted with hexane:dichloromethane:ethyl ether (89:10:1, v/v/v) and contained triacylglycerols. Fraction 1-3 was eluted with hexane:ethyl acetate (95:5, v/v) and contained cholesterol. Fraction 1-4 was eluted with hexane:ethyl acetate (85:15, v/v) and contained diacylglycerols. Fraction 1-5 was eluted with chloroform:methanol (2:1, v/v) and contained monoacylglycerols. All solid phase processes were performed at room temperature.
High Pressure Liquid Chromatography

Extracts from solid phase fractions were evaporated to dryness under nitrogen, resuspended in 250 μl of acetonitrile and subjected to High Pressure Liquid Chromatography (HPLC) in a Waters HPLC system (Waters, Milford, Mass.), following the method described by Lopez et al., Journal of Chromatography B, 760:97 (2001) for separation of monoglycerides and free fatty acids. 50 μl aliquots of samples were injected and separated through a reversed phase Discovery C18 column (3.0×150 mm, particle size 5 μm) (Supelco, Bellefonte, Pa.), using an isocratic elution (running time 10 min) with a mobile phase containing 98.6% acetonitrile and 1.4% acidified water (0.035% formic acid, pH 2.6). Compounds were detected at 215 nm by a Waters ultraviolet detector and fractions collected on a time basis in glass centrifuge tubes. The PPARγ ligand typically eluted between about 2 min. 45 sec. and about 3 min. Collected samples were evaporated under a nitrogen stream and prepared for ligand activity assay.

2. Analytical Methods

High Pressure Liquid Chromatography

The content of the solid phase extracts was analyzed by HPLC using a Waters ultraviolet detector and a Sedere75 Evaporative Light Scattering detector (Sedere, Cranbury, N.J.). Peaks were identified by comparison with retention time of standards. The HPLC conditions are described in the previous paragraph.
Gas Chromatography—Flame Ionization Detector

The fatty acid and plasmalogen content of solid phase extracts was determined by Gas Chromatography with Flame Ionization Detection (GC-FID). Fatty acids from extracts were transmethylated by alkaline hydrolysis, as described by Alvarez and Touchstone (in “Practical Manual on Lipid Analysis: Fatty Acids,” Norell Press, 1991). Dry extracts were resuspended in 0.5 ml of methanolic-base, vortexed and incubated at 100° C. for 3 min, followed by addition of boron trifluoride-methanol (0.5 ml), vortexing, incubation at 100° C. for 1 min, addition of hexane (0.5 ml), vortexing, incubation at 100° C. for 1 min, and addition of 6.5 ml of saturated NaCl. Samples were vortexed and centrifuged at 800×g for 2 min. The hexane upper layer was transferred to a new glass tube.

Plasmalogens from extracts were transmethylated by acidic hydrolysis (Alvarez and Touchstone, “Practical Manual on Lipid Analysis: Fatty Acids,” Norell Press, 1991). In this process, the two initial incubations described for fatty acids were substituted by a 15 min incubation of extracts in the presence of 1 ml of 10N HCl at 100° C. The rest of the transmethylation process is similar.

Methyl esters of fatty acids and plasmalogens were injected in a Hewlett Packard 5890A gas chromatograph. A Supelcowax column of 30 m length and 0.5 mm internal diameter was used. Initial temperature was 150° C. and final temperature 260° C. FID temperature was 300° C. The total running time was 27 min. Peaks were identified by comparison of retention times of standard mixtures.
High Performance Thin Layer Chromatography

To confirm the distribution of the different lipid classes by solid phase fractionation, standards were subjected to solid phase separation and the different fractions analyzed by micro High Performance Thin Layer Chromatography (HPTLC), following the method described by Alvarez and Storey for phospholipid analysis (Mol Reprod Dev, 42:334 (1995)). Solid phase extracts were evaporated to dryness under nitrogen and resuspended in 10 μl of chloroform:methanol (1:1, v/v). Aliquots of 5 μl were applied to 5×5 cm and 200 μm thickness Whatman HP-K silica gel plates (Whatman, Clifton, N.J.), predeveloped in chloroform:methanol (1:1, v/v), developed in Phospholipid Mobile Phase (chloroform:triethylamine:methanol:water, 30:30:34:8, v/v/v/v) to 3.5-4 cm, blow-dried for 30 sec., placed on hot plate (180° C.) for 10 sec., developed again in hexane:ether (100:4.5, v/v) to 4.5 cm, blow-dried for 30 sec., and placed on hot plate for 10 sec. Bands were stained by inmersion in concentrated CuSO₄ solution (100 g of CuSO₄, 95 ml of H₃PO₄ in 1 liter of H₂O), blow-dried for 1 min and developed on hot plate at 180° C. for 3 min. Bands were scanned at 400 nm in the reflectance mode using a Shimadzu CS-9000U spectrodensitometer.

3. Antioxidants

In all experiments, the fractions resulting from the above isolation processes were stored in the presence of butylated hydroxy toluene (BHT), at a concentration of 15 μg/ml of incubation medium (final volume) to minimize the risk of peroxidation.

4. Summary of Fractionation Data

The fractionation experiments using aminopropyl-bonded silica gel columns and following the fractionation protocol described in J. Lipid Res., 26:135 (1985) and J. Chromatogr., 577:142 (1992), consistently show the presence of the bulk of the activity in fraction 1, the neutral lipid fraction. Less activity was also found in fraction 2 (free fatty acids) and even less in fraction 3 (neutral phospholipids: phosphatidylcholine, phosphatidylethanolamine, sphingomyelin and neutral glycolipids). Thus, based on the first solid phase fractionation, acidic phospholipids (fraction 4: phosphatdylglycerol, cardiolipin, phosphatidylinositol, phosphatidylserine and acidic glycosphingolipids) can be ruled out as potential ligands.

Further fractionation of fraction 1, as described above and in J. Lipid Res., 26:135 (1985) and J. Chromatogr., 577:142 (1992), consistently showed significant activity only in the fifth fraction (fraction 1-5). Therefore, a significant part of the ligand activity present in conditioned media, is eluted, first with chloroform:isopropanol 2:1, and second with chloroform:methanol 2:1, showing the solubility-polarity characteristics of monoglycerides (the most polar components of neutral lipids).

4. Chemical Reactions

The effect of several chemical reactions on ligand activity was tested. Extracts of conditioned medium were subjected to enzymatic cleavage by phospholipase A2, phospholipase C and pancreatic lipase, and to a base-catalyzed methanolisis reaction.

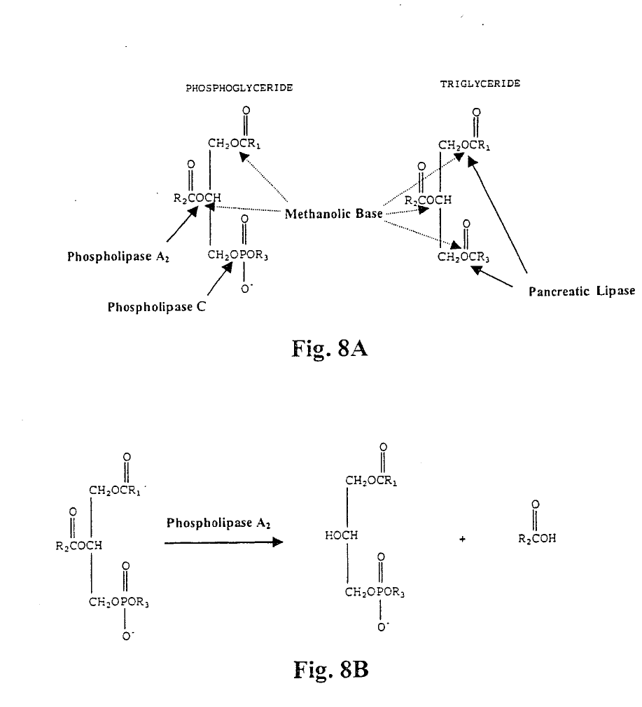

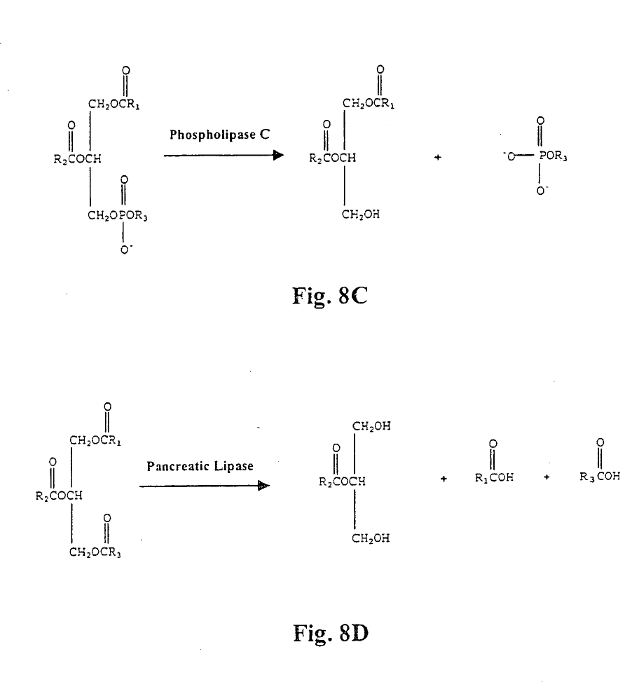

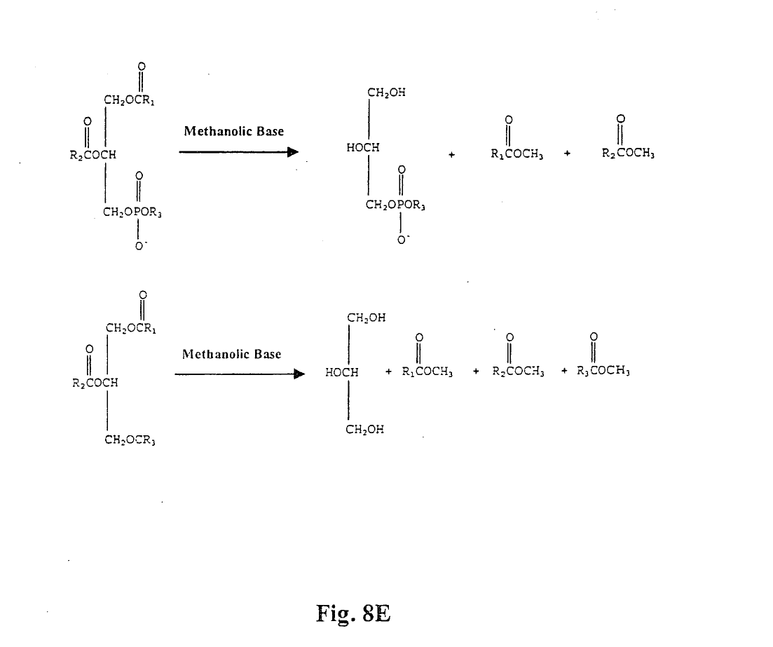

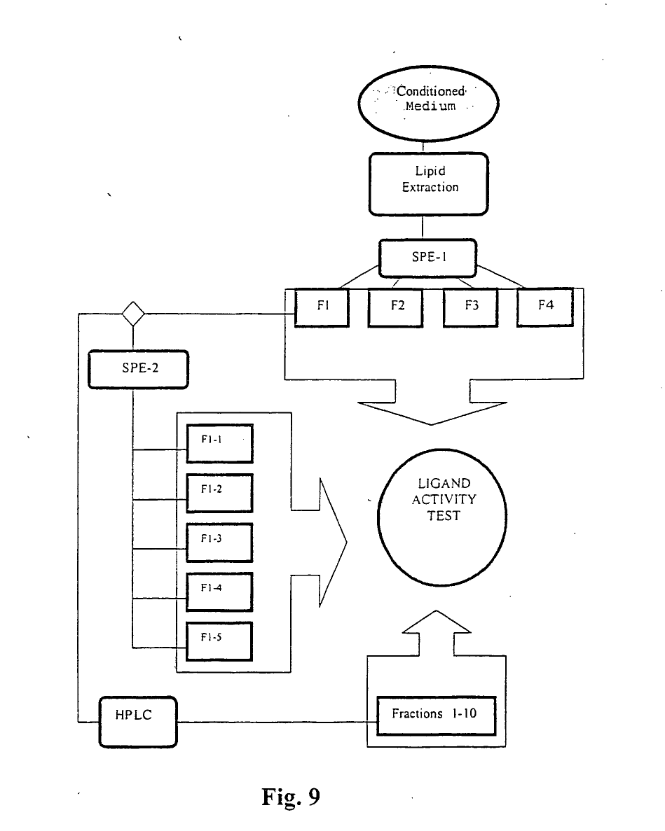

FIG. 8A depicts the cleavage sites of these reactions in both phosphoglycerides and glycerides (mono-, di- or tri-).
Phospholipase A₂

Phospholipase A₂ catalyzes the specific hydrolysis of the fatty acid ester located on the C-2 carbon position of a phosphoglyceride, and yields a lysophosphoglyceride and a free fatty acid molecule, as shown in FIG. 8B.

Lipid extracts from 4 ml aliquots of Conditioned Medium (CM) were resuspended in 1 ml of phosphate buffered saline medium (PBS) by mild sonication in the presence of 1,000 units of phospholipase A₂ from Naja naja venom (Sigma). The mixture was incubated in a water bath at 37° C. for 15 min, and extracted with six volumes of chloroform:methanol (2:1, v/v). A control sample without enzyme was prepared in parallel.
Phospholipase C.

Phospholipase C, classified as a phosphodiesterase, catalyzes the hydrolysis of the ester bond between the diglyceride and the polar head group of a phosphoglyceride, yielding a diglyceride and a phosphate-based compound, as shown in FIG. 8C.

Lipid extracts from 4 ml aliquots of CM were resuspended in 1 ml of PBS by mild sonication in the presence of 100 units of phospholipase C type 1 from C. perfringens (Sigma). The mixture was incubated in a water bath at 37° C. for 5 min, and extracted with six volumes of chloroform:methanol (2:1, v/v). A control sample without enzyme was prepared in parallel.

The results for phospholipase A₂ and C digestions were ambiguous. In one experiment, a partial decrease in ligand activity was found after treatment with both phospholipases A₂ and C. In a second experiment, no decrease was found. Phospholipases are difficult to work with because their activity is dependent on critical micellar concentration.
Pancreatic Lipase

Pancreatic Lipase catalyzes the specific hydrolysis of the fatty acid esters located on the C-1 and C-3 carbon positions of a glyceride (mono-, di- or tri-), and yields either glycerol or a 2-monoglyceride and the free fatty acid molecule(s), as shown in FIG. 8D.

Lipid extracts from 4 ml aliquots of CM were resuspended in 1 ml of PBS by mild sonication in the presence of 1,000 units of Pancreatic Lipase type VI-S from porcine pancreas (Sigma). The mixture was incubated in a water bath at 37° C. for 15 min, and extracted with six volumes of chloroform:methanol (2:1, v/v). A control sample without enzyme was prepared in parallel.

Pancreatic lipase did not decrease ligand activity. This result rules out the ligand being triglycerides, diglycerides and sn-1-(or alpha-) monoglycerides. It does not rule out sn-2- (or beta-) monoglycerides.
Base-catalyzed methanolisis

This reaction transesterifies glycerides, cholesterol esters and phosphoglycerides, yielding methyl esters of fatty acids, glycerol, cholesterol and phosphoglycerol. It also converts free fatty acids to sodium salts. Amide-bound fatty acids, as in sphingolipids, are not affected by this reaction. Aldehydes are not liberated from plasmalogens through this process. The base-catalyzed transesterification of glycerides and phosphoglycerides is depicted in FIG. 8E.

Lipid extracts from 4 ml aliquots of CM were resuspended in 1 ml of toluene and incubated in the presence of 2 ml of methanolic base (Supelco, Bellefonte, Pa.) at 100° C. for 5 min. After cooling down, the mixture was extracted with 1 ml of H₂O and 1 ml of hexane. In the control sample the methanolic base was substituted by toluene.

Base-catalyzed methanolisis abolished ligand activity. This reaction significantly alters the structure of glycerol esters (mono, di and triglycerides), cholesterol esters, phosphoglycerol esters (phospholipids) and transforms free fatty acids into their sodium salts. However, amide-bound fatty acids and aldehyde chains from plasmalogens (phosphoglycerides containing only aldehyde chains) are not affected by this reaction. This would rule out the ligand being a sphingolipid. This would also rule out glycerides and phosphoglycerides containing only aldehyde chains (and not fatty acid chain(s)).

In conclusion, according to the solid phase results, it is reasonable to expect that an endogenous PPARγ ligand, secreted during adipogensis is a monoglyceride. This monoglyceride, according to the pancreatic lipase experiments described above, is an oxidation sensitive, sn-2-monoglyceride.

While this invention has been particularly shown and described with references to preferred embodiments thereof, it will be understood by those skilled in the art that various changes in form and details may be made therein without departing from the scope of the invention encompassed by the appended claims.

1. A method of isolating PPARγ ligand, wherein the ligand is a neutral lipophilic compound comprising;
a) stimulating cells, wherein the cells are present in culture medium, under conditions such that PPARγ ligand is secreted into the culture medium;
b) harvesting the culture medium at about 48 hours after induction of differentiation; and
c) isolating neutral lipophilic compounds from the culture medium;
thereby isolating PPARγ ligand.

2. A method of claim 1, wherein the cells are selected from the group consisting of: disaggregated fibroblast-like cells, pre-adipocytes, primary adipocytes and adipose tissue.

3. A method of claim 2, wherein the cells are 3T3-L1 cells.

4. A method of claim 1, wherein the cells are stimulated such that intracellular cAMP levels are elevated.

5. A method of claim 4, wherein said compound is selected from the group consisting of: methyl iso-butyl xanthine, 8-bromo-cAMP and Forskolin.

6. A method of claim 4, wherein said compound is methyl iso-butyl xanthine at a concentration of about 0.05 to about 5 mM.

7. A method of claim 1, where in the cells are stimulated to differentiate into adipocytes.

8. A method of claim 7, wherein the cells are contacted with a mixture of methyl iso-butyl xanthine at a concentration of about 0.05 to about 5.0 mM, dexamethasone at a concentration of about 0.04 to about 4.0 μg/ml and insulin at a concentration of about 0.5 to about 50 μg/ml.

9. A method of claim 1, wherein the neutral lipophilic compounds are isolated from the culture medium by;
i) extracting the harvested medium with six volumes of a mixture of chloroform and methanol at a ratio of about 2 parts to about 1 part by volume;
ii) loading the organic phase of step i) onto an aminopropyl column, and eluting bound lipophilic compounds with a mixture of chloroform and isopropanol at a ratio of about 2 parts to about 1 part by volume; and
iii) loading the eluted fraction of step ii) onto a second aminopropyl column, and eluting bound lipophilic compounds with a mixture of chloroform and methanol at a ratio of about 2 parts to one part by volume;
thereby isolating PPARγ ligand.

10. A method of claim 9, further comprising subjecting the eluted fraction of step iii), to HPLC, wherein the PPARγ ligand elutes from the HPLC column between about 2 min. 45 sec. and about 3 min. 15 sec.

11. The PPARγ ligand of claim 10.

12. The PPARγ ligand of claim 11, wherein the ligand comprises a monoglyceride.

13. A method of isolating PPARγ ligand, wherein the ligand is a neutral lipophilic compound, comprising;
a) inducing 3T3-L1 cells to differentiate into adipocytes, wherein the 3T3-L1 cells are contacted with a methyl iso-butyl xanthine at a concentration of about 0.05 to about 5.0 mM, dexamethasone at a concentration of about 0.04 μg/ml to about 4.0 μg/ml, and insulin at a concentration of about 0.5 to about 50 μg/ml, such that PPARγ ligand is secreted into the culture medium;
b) harvesting the culture medium at about 48 hours after induction of differentiation; and
c) isolating neutral lipophilic compounds from the culture medium by;
i) extracting the harvested medium with six volumes of a mixture of chloroform and methanol at a ratio of about 2 parts to about 1 part by volume;
ii) loading the organic phase of step i) onto an aminopropyl column, and eluting the PPARg ligand with a mixture of chloroform and isopropanol at a ratio of about 2 parts to about 1 part by volume; and
iii) loading the eluted fraction of step ii) onto a second aminopropyl column, and eluting the PPARg ligand with a mixture of chloroform and methanol at a ratio of about 2 parts to one part by volume;
thereby isolating PPARγ ligand.

14. A method of claim 13, further comprising subjecting the eluted fraction of step iii), to HPLC, wherein the PPARγ ligand elutes from the HPLC column between about 2 min. 45 sec. and about 3 min. 15 sec.

15. The PPARγ ligand of claim 14.

16. The PPARγ ligand of claim 15, wherein the ligand comprises a monoglyceride.

17. A composition comprising a PPARγ ligand, wherein the ligand is a neutral lipophilic compound and is isolated using the method of claim 1, and a pharmaceutical carrier.

18. A method of increasing PPARγ activity in cells of interest, comprising contacting a the cells of interest with isolated PPARγ ligand, wherein said ligand is isolated by;
a) inducing cultured cells to differentiate into adipocytes, such that PPARγ ligand is secreted into the culture medium;
b) harvesting the culture medium at about 48 hours after induction of differentiation; and
c) isolating neutral lipophilic compounds from the culture medium; thereby isolating the PPARγ ligand;
such that PPARγ activity in the cells of interest is increased.

19. A method of claim 18, wherein the cultured cells are 3T3-L1 cells.

20. A method of claim 18, wherein the neutral lipophilic compounds are isolated from the culture medium by;
i) extracting the harvested medium with six volumes of a mixture of chloroform and methanol at a ratio of about 2 parts to about 1 part by volume;
ii) loading the organic phase of step i) onto an aminopropyl column, and eluting bound lipophilic compounds with a mixture of chloroform and isopropanol at a ratio of about 2 parts to about 1 part by volume; and
iii) loading the eluted fraction of step ii) onto a second aminopropyl column, and eluting bound lipophilic compounds with a mixture of chloroform and methanol at a ratio of about 2 parts to one part by volume.
